(19) United States
(12) Patent Application Publication
(10) Pub. No.: US 2023/0086831 A1
(43) Pub. Date: Mar. 23, 2023

(54) PRODUCTION METHOD OF RETINAL PIGMENT EPITHELIAL CELL

(71) Applicant: RIKEN, Wako-Shi, Saitama (JP)

(72) Inventors: Masayo Takahashi, Wako-Shi, Saitama (JP); Cody West Kime, Wako-Shi, Saitama (JP); Ivo Ngundu Woogeng, Wako-Shi, Saitama (JP)

(21) Appl. No.: 17/802,577

(22) PCT Filed: Feb. 26, 2021
(86) PCT No.: PCT/JP2021/007320

§ 371 Date: Aug. 26, 2022

(30) Foreign Application Priority Data
Feb. 28, 2020 (JP) 2020-033848

(51) Int. Cl. C12N 5/079; A61K 35/30

(52) U.S. Cl. CPC C12N 5/0621; A61K 35/30; C12N 2501/115; C12N 2501/15; C12N 2501/608; C12N 2510/00

(57) ABSTRACT

The present invention aims to provide a method for preparing an RPE cell, and an RPE cell prepared by the method, and a reagent for producing an RPE cell which is suitable for the method. The method of the present invention includes the following step: a step of introducing, as exogeneous factors, MITF (Microphthalmia-Associated Transcription Factor) gene or an expression product thereof, OTX2 (Orthodenticle homeobox 2) gene or an expression product thereof, LIN28 gene or an expression product thereof, and L-MYC gene or an expression product thereof into a mammalian somatic cell.

### TECHNICAL FIELD

The present invention relates to a method for producing a retinal pigment epithelial cell. More particularly, the present invention relates to a method for producing a retinal pigment epithelial cell by direct conversion (direct-reprogramming), and a reagent for converting a somatic cell to a retinal pigment epithelial cell and the like.

### BACKGROUND ART

A treatment method including producing retinal pigment epithelial (RPE) cells from iPS cells and administering same to patients with wet age-related macular degeneration requires about 6 months of culture period and is extremely costly. It is realistic to use iPS stock for iPS cells, in which case allotransplantation is performed. There is a need for a method that can shorten the culture period to reduce costs and can treat especially patients requiring autologous transplantation.

A method for producing RPE-like cells by forcibly expressing plural transcription factors in fibroblasts has been reported (PTL 1, NPL 1). RPE-like cells are produced by overexpressing at least 4 kinds of transcription factors which are selected from 10 kinds of transcription factors in the former and 8 kinds of transcription factors in the latter; however, a stable cell line has not been established. Each combination is different from that in the present invention.

### CITATION LIST

### Patent Literature

[PTL 1] US2017/0002319A1

### Non Patent Literature

[NPL 1] Zhang, Protein Cell 2014, 5(1); 48-58

### SUMMARY OF INVENTION

### Technical Problem

The present invention aims to provide a method for producing an RPE cell capable of autologous transplantation to patients for whom transplantation of RPE cells is effective, for example, patients with wet age-related macular degeneration, an RPE cell produced by the method, and a reagent (e.g., vector) for producing an RPE cell suitable for the method.

### Solution to Problem

In view of the above-mentioned problem, the present inventors conducted intensive studies based on the findings to date. To be specific, they tried to produce an RPE cell from a somatic cell by a direct conversion (direct reprogramming) technique. Direct reprogramming can be achieved by transducing a gene that encodes a transcription factor that plays a key role in the differentiation stage of the target cell or a gene that promotes reprogramming of a somatic cell. The present inventors have found that a somatic cell can be directly converted to an RPE cell by introducing some genes, established the conditions of the conversion, and analyzed the phenotype and function of the obtained directly-converted RPE cell, which resulted in the completion of the present invention.

Therefore, the present invention provides the following.

(1) A method for producing a retinal pigment epithelial cell, comprising a step of introducing, as exogeneous factors,

an MITF (Microphthalmia-Associated Transcription Factor) gene or an expression product thereof,

an OTX2 (Orthodenticle homeobox 2) gene or an expression product thereof,

a LIN28 gene or an expression product thereof, and

an L-MYC gene or an expression product thereof

into a mammalian somatic cell.

(2) The method of the above-mentioned (1), further comprising a step of introducing a CRX (cone-rod homeobox) gene or an expression product thereof as the exogeneous factor.

(3) The method of the above-mentioned (1) or (2), comprising 3 stages (phases) of a period of overexpressing the introduced exogeneous factors in the somatic cell to yield genomic plasticity (phase 1), a period of converting the somatic cell identity to an RPE cell (phase 2), and a period of maturing the converted RPE cell (phase 3).

(4) The method of the above-mentioned (3), comprising using a medium comprising bFGF and 2-ME in phase 1.

(5) The method of the above-mentioned (3), comprising using a medium comprising Chetomin and Nicotinamide in a part of the period of phase 2.

(6) The method of the above-mentioned (3), comprising using a medium comprising SB431542 and bFGF in phase 3.

(7) The method of any of the above-mentioned (1) to (6), wherein the somatic cell is a human fibroblast.

(8) An RPE cell produced by the method of any of the above-mentioned (1) to (7).

(9) An RPE cell derived from a mammalian somatic cell, and comprising, as exogeneous factors,

an MITF gene or an expression product thereof,

an OTX2 gene or an expression product thereof,

a LIN28 gene or an expression product thereof, and

an L-MYC gene or an expression product thereof.

(10) The cell of the above-mentioned (9), further comprising a CRX gene or an expression product thereof as the exogeneous factor.

(11) The cell of the above-mentioned (9) or (10), wherein the somatic cell is a human fibroblast.

(12) A reagent for directly producing a retinal pigment epithelial cell from a somatic cell, comprising

an MITF gene or an expression product thereof,

an OTX2 gene or an expression product thereof,

a LIN28 gene or an expression product thereof, and

an L-MYC gene or an expression product thereof.

(13) The reagent of the above-mentioned (12), further comprising a CRX gene or an expression product thereof.

(14) The reagent of the above-mentioned (12) or (13), wherein the somatic cell is a human fibroblast.

### Advantageous Effects of Invention

The current state-of-the-art approach to generate patient-specific Retinal Pigment Epithelium (RPE) is to induce their somatic cells into pluripotent stem (iPS) cells. The skilled person must then expand and qualify those iPS cells, and then differentiate those iPS cells into RPE cells, requiring over 180 days of cell culture, which is prohibitively expensive for practical medicine. The technology proposed by the present invention drastically reduces the somatic to RPE cell transition by rapidly changing somatic donor cells to cells with RPE-like identity within 2-3 weeks, and abundant stable early RPE cells within 40-60 days.

The present invention utilizes specialized cell media conditions and overexpression of key transcription factors to rapidly and efficiently generate RPE cells from easily available patient-cells, such as skin or blood. The costs of patient-specific cells for medicine or research are almost completely derived from the labor and materials required each day, and the current state-of-the-art production of patient-specific RPE cells is not cost-effective for patients or scientists. The present invention drastically reduces the costs of patient-specific autologous RPE cells to enable a realistic cell product for regular medical and research applications.

The present invention is compared to current state-of-the art iPS cell technology derived RPE (FIG. 1).

### BRIEF DESCRIPTION OF DRAWINGS

FIG. 1 shows that the comparison of the present invention and the current state-of-the art.

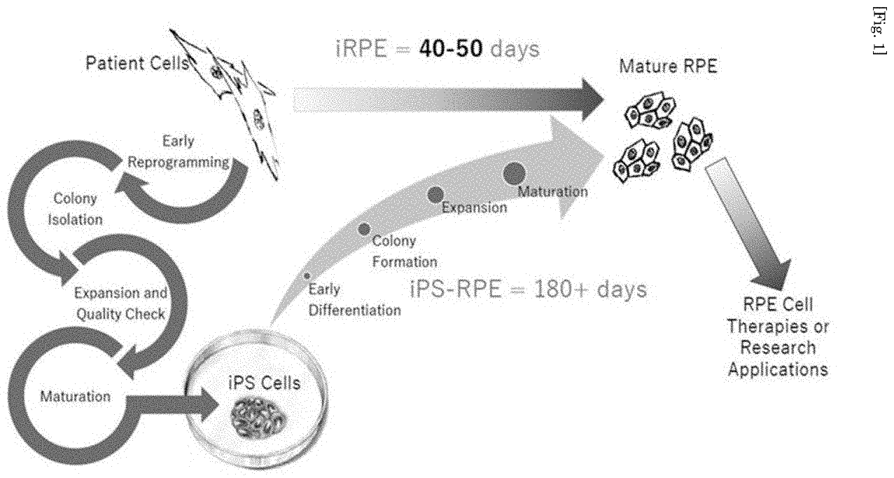

FIG. 2 shows the proposed model of how overexpression of human exogenous transcription factors first induces intermediate cell plasticity and secondarily reinforces development of the RPE cell identity.

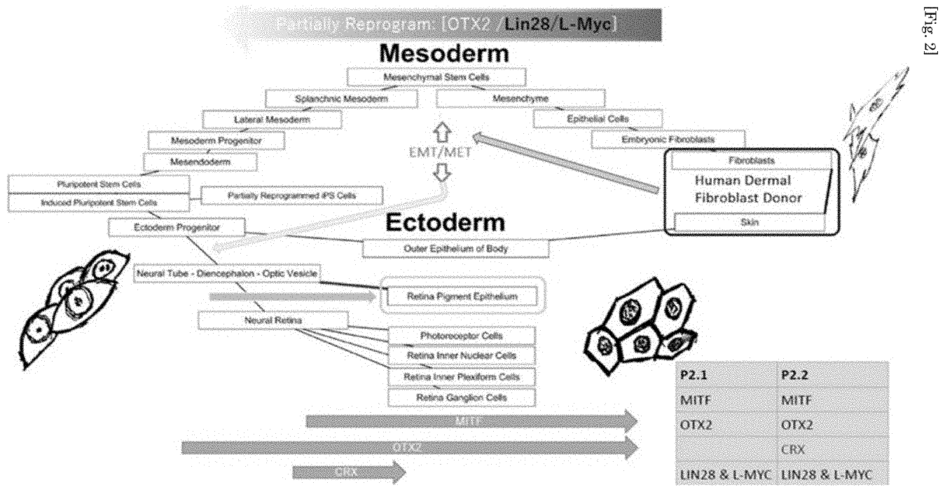

FIG. 3 shows the three phases of reprogramming media and discrete timing of materials that work with the overexpression of reprogramming factors (see, FIG. 2) to quickly convert cells to RPE cell identity.

FIG. 4 The reprogramming of human foreskin fibroblasts to iRPE (induced RPE) cells is imaged over time and a single colony production of RPE cells is obviated within 30 days. Cells can be subcultured thereafter similar to RPE cells with live maturation reporter for RPE gene Best1 (BEST1::EGFP).

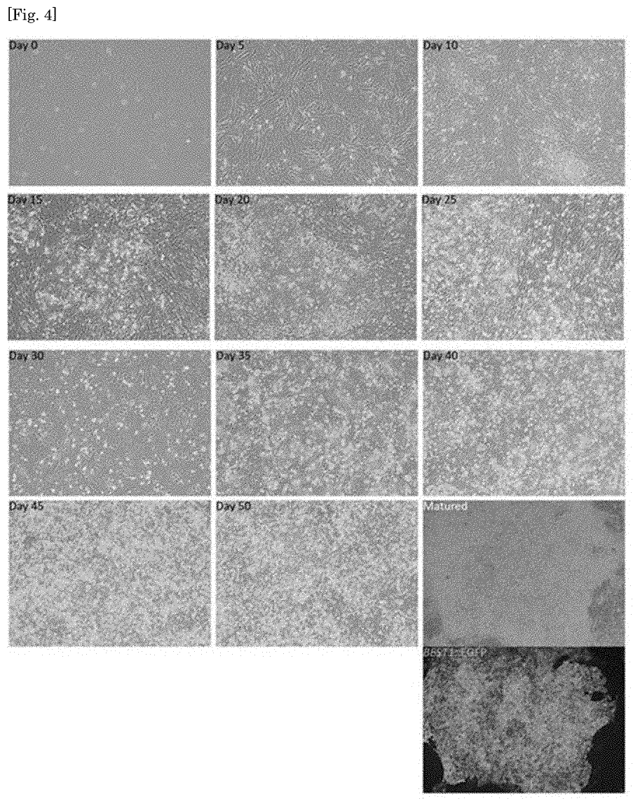

FIG. 5 Human iRPE cells induced by the present invention system express critical RPE genes RPE65 and ZO-1 (TJP1), as examined by fluorescent immunocytochemistry microscopy. (A) DNA (Hochest 33342), (B) BEST1 (Best1::EGFP reporter), (C) RPE65 (immunocytochemistry), (D) ZO-1 (immunocytochemistry), (E) Merged image of A, B, C, D.

FIG. 6 is a graph showing the results of the increase in colony average diameter formation due to the addition of CRX. The Human iRPE cells can be induced without CRX, but colony formation efficiency, size, and proliferation, are greatly increased with the addition of CRX. Data are presented as the mean±Max/Min. Reprogramming with or without CRX is also measured by colony numbers are counted on day 9 and shown in Table 4.

FIG. 7 iRPE cell reprogramming of human fibroblasts induces RPE-like cells with live maturation BEST1::EGFP reporter activity among pigmented cobblestone cells resembling mature RPE.

FIG. 8 iRPE cell reprogramming of human fibroblasts may be improved with Chetomin and Nicotinamide treatments late in Phase 2 of reprogramming. In these two panels, the same mixed population of iRPE reprogramming was treated with or without Chetomin and Nicotinamide, and then carried into Phase 3 where the pigmentation intensity and RPE-cell distinction may be improved. (upper panel) without Chetomin and Nicotinamide, (lower panel) with Chetomin and Nicotinamide.

FIG. 9 Stable cultured human iRPE cells were transplanted into immunocompromised albino rat eyes with high survival and integration. Fluorescent imaging upon dissection after 4.5 months shows that several large areas of the transplanted human iRPE cells expressed the BEST1::EGFP live maturation reporter gene transfer. (upper panel) Photograph, (lower panel) BEST1::EGFP.

FIG. 10 Stable cultured human iRPE cells were transplanted into immunocompromised albino rat eyes with high survival and integration. Cryosection of dissected tissues after 4.5 months was performed. Hematoxylin and Eosin staining shows that several human iRPE cells have become pigmented cells that have grown, matured, and interfaced with neural retina, including integration and sheet formation in the correct RPE-layer.

### DESCRIPTION OF EMBODIMENTS

The present invention is explained in the following. Unless particularly indicated, the terms used in the present specification have the meanings generally used in the field.

The present invention relates to a method for producing a retinal pigment epithelial (RPE) cell by converting a mammalian differentiated somatic cell to an RPE cell. The “converting” means changing a somatic cell into a desired RPE cell. One of the characteristics of the method of the present invention is a method for converting a somatic cell to an RPE cell, which is called “direct reprogramming” or “direct conversion”, without a step of completely reprogramming a cell represented by production of iPS cell.

Induction into an RPE cell could theoretically be performed both in vitro and in vivo.

RPE Cells

The “retinal pigment epithelial cell” in the present invention refers to an epithelial cell constituting the retinal pigment epithelium, and a progenitor cell thereof. Whether a retinal pigment epithelial cell or not can be confirmed by, for example, expression of cell markers (RPE65, CRALBP, MERTK, BEST1 etc.), cell forms (intracellular melanin pigment deposition, polygonal and flat cell form, formation of polygonal actin bundle etc.) and the like. The progenitor cell of retinal pigment epithelial cell means a cell directed to be induced to differentiate into retinal cell, and whether a progenitor cell or not can be confirmed by expression of cell markers (Mitf, Pax6, Rx, Crx etc.) and the like. These functional evaluation and confirmation operations can be performed by those of ordinary skill in the art by setting appropriate conditions.

The RPE cell induced and prepared by the present invention is also referred to as an induced RPE cell (iRPE cell).

Somatic Cell

The somatic cell to be the target of direct reprogramming in the present invention is not particularly limited as long as RPE cell can be induced and may be derived from a mammal. When iRPE cell is transplanted to a living organism, a somatic cell (autologous cell) derived from a test subject that receives transplantation is preferably used to reduce the risk of infection, rejection response and the like. Depending on the purpose, however, RPE cells prepared in advance from somatic cells of other people or other animals, not autologous cells, can be used for transplantation.

In the present specification, examples of mammals include mice, rats, hamsters, humans, dogs, cats, monkeys, rabbits, cows, horses, pigs, and the like, particularly humans.

As the somatic cell, a somatic cell that can be easily collected from a living body can be used. Examples thereof include fibroblasts, keratinocytes, oral mucosal epithelial cells, nasal mucosal epithelial cells, respiratory mucosal epithelial cells, gastric mucosal epithelial cells, intestinal mucosal epithelial cells, vascular endothelial cells, smooth muscle cells, adipocytes, gingival cells (gingival fibroblasts and gingival epithelial cells), dental pulp cells, periodontal ligament cells, bone marrow cells, bone marrow-derived interstitial cells, leukocytes, lymphocytes, conjunctival epithelial cells, and osteoclasts, preferably fibroblasts, keratinocytes, oral mucosal epithelial cells, gingival cells, leukocytes, and lymphocytes. In the present invention, the above-mentioned cell collected from a living body is preferably used.

Gene or Expression Product Thereof

In the method of the present invention, as exogeneous factors, MITF (Microphthalmia-Associated Transcription Factor) gene or an expression product thereof, OTX2 (Orthodenticle homeobox 2) gene or an expression product thereof, LIN28 gene or an expression product thereof, and L-MYC gene or an expression product thereof are introduced into somatic cells. When desired, CRX (cone-rod homeobox) gene or an expression product thereof may be introduced as the exogeneous factor, and introduction thereof is preferable from the aspect of colony formation efficiency. As used herein, the “expression product” is, for example, mRNA or protein of MITF gene, OTX2 gene, LIN28 gene, L-MYC gene or CRX gene.

All of the above genes are highly conserved in vertebrates. In this specification, they refer to genes including homologues, unless a specific animal name is described. The genes further include genes having functions equivalent to those of wild-type gene products, even when the genes include mutations including polymorphisms.

For example, nucleotide sequences of human (Homo sapiens) MITF gene, OTX2 gene, LIN28 gene, L-MYC (MYCL) gene and CRX gene, and amino acid sequences of proteins encoded by these sequences have been registered at GenBank provided by the National Center for Biotechnology Information (NCBI). As an embodiment, the following accession numbers are exemplified (it should be understood that when multiple revisions have been registered, each number refers to the latest revision):

Human MITF gene: e.g., AB006909.1,

Human MITF protein: e.g., BAA32288.1,

Human OTX2 gene: e.g., NM_021728.4,

Human OTX2 protein: e.g., NP_068374.1,

Human LIN28 gene: e.g., NM_024674.6,

Human LIN28 protein: e.g., NP_078950.1,

Human L-MYC gene: e.g., NM_001033081.3,

Human L-MYC protein: e.g., NP_001028253.1,

Human CRX gene: e.g., NM_000554.6,

Human CRX protein: e.g., NP_000545.1.

It is clear that other sequences can be used as long as they correspond to the same or similarly functional gene and/or proteins, respectively.

### INTRODUCTION

The method of the present invention can be performed according to a known reprogramming method, except that specific genes are selected. To be specific, a desired gene as the exogeneous factor (hereinafter to be simply referred to as desired gene) is introduced and expressed in a targeting somatic cell.

As a method of introducing a gene, there can also be used, for example, a method involving infection with a viral vector, such as a retrovirus vector, an adenovirus vector, a lentivirus vector, an adeno-associated virus vector, a herpes virus vector, or a Sendai virus vector; and in the case of introduction of a gene and an expression product thereof, a method involving transfection with a plasmid vector, an episomal vector, or a gene expression product (mRNA, protein) by a non-viral vector, such as a cationic liposome, a cationic polymer, or electroporation. Alternatively, mRNA can also be introduced. Genome-editing technology such as CRISPR/Cas9 system (CRISPR targeted transactivation) can also be utilized.

A preferable one embodiment is a method for introducing an expression cassette designed to control expression of the desired gene in a period specific manner in response to the external stimulation. Such expression cassette is a nucleic acid construct containing at least a promoter capable of inducing expression of a gene in the downstream in response to an external stimulation, and a desired gene whose expression is controlled by the promoter.

The promoter is not particularly limited as long as it is a promoter capable of inducing expression of the gene in the downstream in response to the external stimulation. Examples thereof include, when the external stimulation is the presence of a tetracycline antibiotic (tetracycline, tetracycline derivative such as doxycycline and the like), a promoter capable of inducing expression of the gene in the downstream by the binding of a complex of a tetracycline antibiotic and a tetracycline transactivator.

The expression cassette may contain, where necessary, enhancer, silencer, selection marker gene (e.g., drug resistance gene such as neomycin resistance gene and the like), SV40 replication origin and the like.

Thus, the external stimulation includes culture in the presence or absence of a drug. For example, expression of the desired gene is controlled in the presence of doxycycline and using a doxycycline expression induction system (e.g., doxycycline expression induction system).

The doxycycline expression induction system may be a commercially available one (e.g., TAKARA, Clontech etc.), or produced by a method described in a known document.

A method for introducing the aforementioned expression cassette into a somatic cell is not particularly limited and a known method can be appropriately selected and used.

For example, the aforementioned expression cassette is inserted into a suitable expression vector, and can be introduced by a known transformation method such as virus infection using a virus vector such as retrovirus vector, adenovirus vector and the like, lipofection method, liposome method, electroporation method, calcium phosphate method, DEAE dextran method, microinjection method and the like.

Examples of the expression vector include virus vectors such as lentivirus, retrovirus, herpes virus, adenovirus, adeno-associated virus, Sendai virus and the like, and animal cell expression plasmid. From the aspect of introduction efficiency, lentivirus is preferable.

As for the aforementioned external stimulation, those of ordinary skill in the art can appropriately adjust the amount (level) of the stimulation to be added in consideration of the kind and the like of the aforementioned promoter to be utilized and in consideration of the time of addition. For example, when the external stimulation is the presence of doxycycline, a preferable addition concentration of doxycycline is 0.1-2 μg/ml, more preferably 0.5-1 μg/ml. In one embodiment, the amount to be added is 1 μg/ml in phase 1 and phase 2, and then reduced briefly to 0.5 μg/ml in phase 3 where it is soon completely removed.

An external stimulation such as doxycycline and the like are allowed to exist in a somatic cell, into which a desired gene has been introduced, for 60 days, preferably at least 50 days, after the desired gene transfer. As a result, the somatic cell is induced into an RPE cell and can be matured in phase 3.

Culture

In the method of the present invention, a mammalian differentiated somatic cell can be cultured in a medium after introduction of the gene. For example, it is a preferable embodiment wherein RPE is induced (prepared) in vitro.

Culture can be performed in a container appropriate for storing cells and medium. In the case of adhesion culture, a cell adhesive culture vessel, for example, a culture vessel after a coating treatment with an extracellular matrix etc. (e.g., poly-D-lysine, laminin, fibronectin, iMatrix511(product name)), is preferably used. The culture conditions of the adhesion culture such as culture temperature, CO₂ concentration, and O₂ concentration can be determined as appropriate. In this case, the cells may be cultured in the presence of a serum, a known growth factor, and additives and chemical substances that promote growth. Examples of the known growth factor include EGF, FGF and the like. Examples of the additives that promote growth include N2 supplement (Invitrogen), B27 supplement (Invitrogen) and the like.

The period of culture is not particularly limited as long as the effect of the present invention is not impaired. For example, it can be set to about 30 days, or 40 days, or 50 days, or 60 days, or 70 days, as necessary. Where necessary, the medium can be changed, and it is preferable to change to a medium with components appropriately adjusted according to the stage (see the following “Medium”).

Medium

The medium used in the method of the present invention is not particularly limited. Usual liquid media such as DMEM (Dulbecco's Modified Eagle's Medium), EMEM (Eagle's Minimal Essential Medium), αMEM (alpha Modified Minimum Essential Medium), Ham's F-12 (Nutrient Mixture F-12 Ham) and the like can be used. If necessary, serum components (Fetal Bovine Serum (FBS), Human Serum (HS)), serum replacement (SR), antibiotics, such as streptomycin and penicillin, non-essential amino acids (NEAA), and similar components can be added.

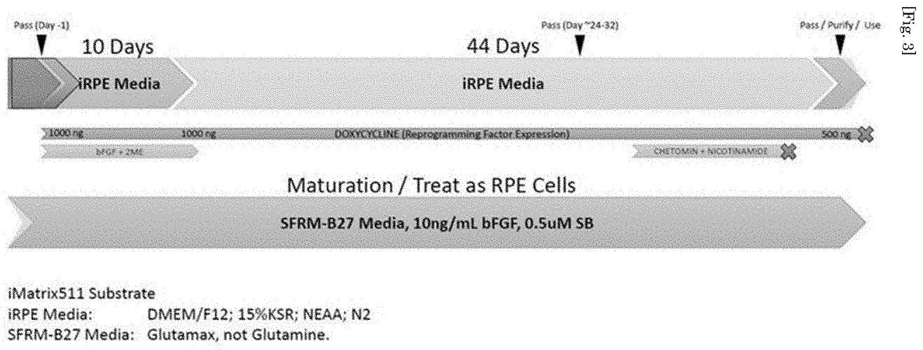

In view of the high preparation efficiency of RPE cell by the method of the present invention, it is preferable to divide the preparation process of RPE cell into three stages (phase 1, phase 2, phase 3), and use a medium suitable for each phase (see FIG. 3). These media are sometimes referred to as phase 1 medium, phase 2 medium, and phase 3 medium for convenience.

Phase 1 medium is a medium to be used for a period of overexpression of the introduced desired gene in a somatic cell, and typically used for 8-10 days after gene transfer. From the aspect of sustaining early cell proliferation, the medium preferably contains bFGF and 2-ME. A preferable addition concentration of bFGF is 1-100 ng/ml, more preferably about 10 ng/ml. A preferable addition concentration of 2-ME is 10-100 μM, more preferably about 55 μM.

Phase 2 medium is a medium to be used for a period of conversion of the cell into which the desired gene has been introduced after phase 1 to an RPE cell, and typically used for 34-38 days after phase 1. The medium preferably contains Chetomin and Nicotinamide so that undesirable cells can be eliminated. A preferable addition concentration of Chetomin is 10-100 nM, more preferably 40-80 nM. A preferable addition concentration of Nicotinamide is 1-50 mM, more preferably 5-10 mM.

Phase 3 medium is a medium to be used for a period of maturation of the converted RPE cells after phase 2, and typically used for 7-12 days after phase 2. From the aspect of common RPE cell maturation materials, the medium preferably contains SB431542 and bFGF. A preferable addition concentration of SB431542 is 0.5-1 μM, more preferably about 0.5 μM. A preferable addition concentration of bFGF is 1-100 ng/ml, more preferably about 10 ng/ml.

Production (Induction)

Thus, an RPE cell is produced from a somatic cell. Whether the cell is a retinal pigment epithelial cell can be confirmed by those of ordinary skill in the art based on, for example, expression of cell markers (RPE65, Mitf and the like), presence of melanin granule, characteristic cell form of polygon and so on.

The induced RPE cell (iRPE cell) contains exogenous MITF gene, OTX2 gene, LIN28 gene and L-MYC gene (preferably, also CRX gene). The term “exogenous” as used herein means an embodiment of a gene or an expression product thereof that is introduced mainly by the above introduction means and that is different from native embodiment.

The iRPE cell may be obtained as a mixture with a cell other than iRPE cell (e.g., original somatic cell). In this case, the iRPE cell can be separated from the cell other than iRPE cell as necessary. The means for separation is not particularly limited. For example, they can be separated using a cell sorter or magnetic beads.

iRPE cells produced by the present invention have characteristics extremely similar to those of retinal pigment epithelial cells in the body. Therefore, it can also be used for screening for a therapeutic drug for a disease due to a disorder of retinal cells, or a transplantation material for cell therapy, a material for the study of diseases or a drug discovery material for a therapeutic drug for a cell damage due to other etiology. In addition, they can be utilized for the study of toxicity such as phototoxicity, toxicity test and so on in the toxicity and drug efficacy evaluation of chemical substances and so on. Examples of the disease due to a disorder of retinal cells include organic mercury poisoning, chloroquine retinopathy, retinitis pigmentosa, age-related macular degeneration, glaucoma, diabetic retinopathy, neonatal retinopathy, and so on.

iRPE cells produced by the present invention can be used as a retinal pigment epithelial cell for transplantation, which is used for supplementing a damaged cell or disordered tissue itself in a cell damage state (e.g., used for transplantation operation) and so on.

Reagent

As mentioned above, RPE cell can be induced by introducing, as exogeneous factors, MITF gene or an expression product thereof, OTX2 gene or an expression product thereof, LIN28 gene or an expression product thereof and L-MYC gene or an expression product thereof, and preferably CRX gene or an expression product thereof in addition thereto, into a somatic cell. Therefore, the present invention further provides a reagent for producing an RPE cell from a somatic cell, containing MITF gene or an expression product thereof, OTX2 gene or an expression product thereof, LIN28 gene or an expression product thereof and L-MYC gene or an expression product thereof, and preferably CRX gene or an expression product thereof in addition thereto. The somatic cell here is preferably a fibroblast.

The reagent specifically includes, as a form in which the above-mentioned genes can be introduced into the somatic cell, a vector having the above-mentioned genes incorporated therein. The above-mentioned genes may be each incorporated in different vectors, or two or more kinds of genes may be simultaneously incorporated in one vector.

The kind and the like of the vector that can be used are as described above.

The present invention is explained in detail in the following by referring to Examples which are not to be construed as limitative. The reagents and materials to be used are commercially available unless particularly limited.

### EXAMPLES

### Example 1: Induction of RPE Cells

(Phase 1)

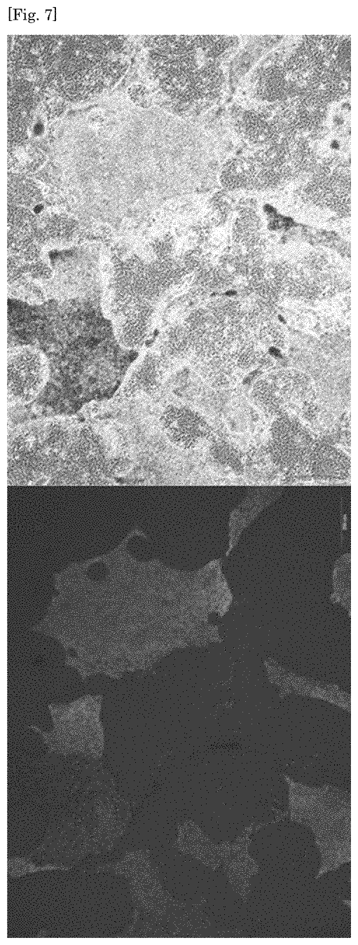

Human somatic cells (fibroblasts; ATCC Cat #CRL2522) were cultured and expanded in standard media and methods until reprogramming was desired. To prepare for reprogramming, the fibroblasts were transduced with the lentiviral dox-inducible conditional gene expression system and dox-inducible lentivirii for MITF, OTX2, LIN28, L-MYC, and CRX. To initiate reprogramming, fibroblasts were plated at 100-150×10³ cells/well on 6 well (6W) plate wells coated with iMatrix511. The following day, reprogramming media Phase 1 (Phase 1 medium) was applied with daily overexpression of human exogenous transcription factors (MITF, OTX2, LIN28, L-MYC and CRX) by using lentiviral dox-inducible system. The reprogramming factor overexpression was maintained until it was gradually removed later in early Phase 3 when iRPE become stable (FIG. 7). The culture using the Phase 1 medium was maintained for 8-10 days.

(Phase 2)

Next, Phase 2 medium was applied bi-daily (every two days). At approximately day 24-32, each 6W plate well of reprogramming was replated in bulk in Phase 2 medium onto iMatrix511 coated 6W plates at 400-600×10³ cells/well, in one or more wells (typically 2 wells). Alternatively, 12 well (12W) plates can be used with 200-300×10³ cells/well. Alternatively, iRPE colonies can be isolated to individual iMatrix511 coated 96 well (96W) plate well, although bulk passage is advisable.

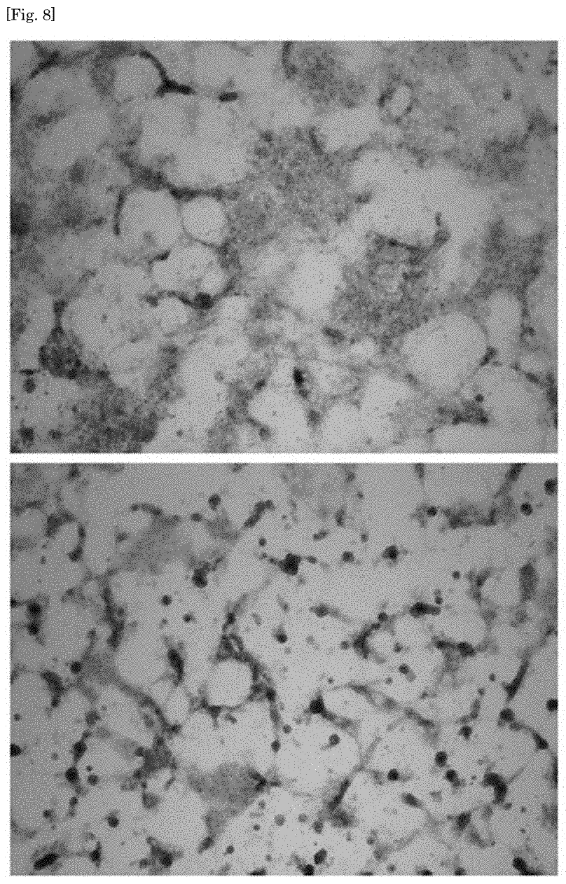

After replating, reprogramming was continued for approximately one more week in Phase 2 medium. Then, an optional treatment of 40-80 nM Chetomin and 5-10 mM Nicotinamide can be added for 6-12 days to improve RPE cell quality (FIG. 8), or the culture using Phase 2 medium can be continued bi-daily for the same period.

The culture using Phase 2 medium was continued for 2 more days (one media change) after removal of Chetomin and Nicotinamide.

(Phase 3)

Phase 3 medium was applied bi-daily to cause RPE-like maturation and increase polarization and pigmentation. For 7-12 days, the overexpression of the exogenous MITF, OTX2, LIN28, L-MYC and CRX was gradually reduced until they were no longer added. During this process of losing reprogramming exogenous, and culture thereafter, the quality of iRPE and undesired cells became distinct (FIG. 7).

The culture using Phase 3 medium was maintained and pigmented cells and/or those with continued BEST1::EGFP reporter expression were considered stably induced into RPE (iRPE). These reprogrammed and differentiated iRPE cells can then be purified, subcultured, examined, and transplanted with standard RPE culture methods.

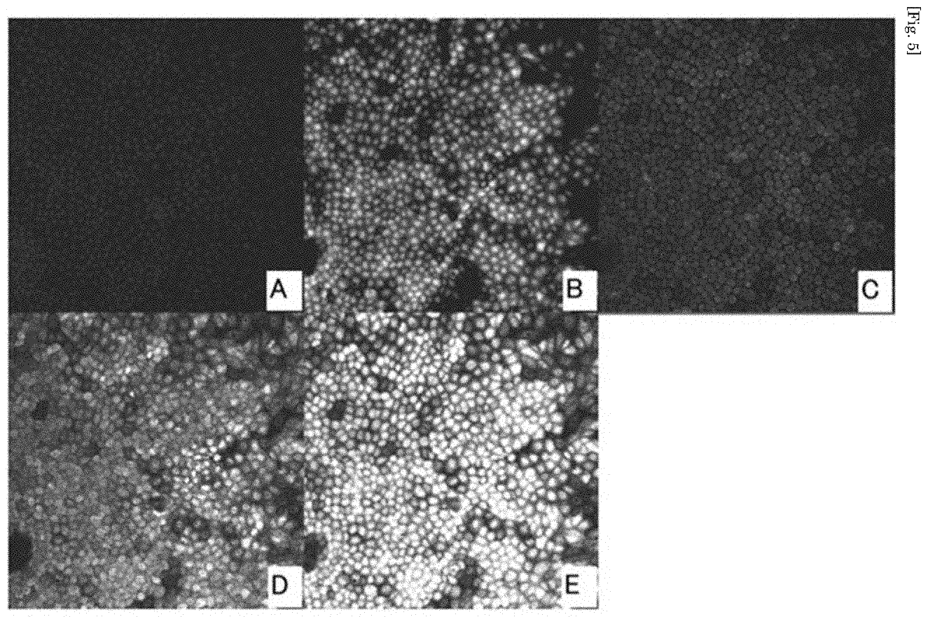

The reprogramming of human foreskin fibroblasts to iRPE cell was observed over time. The expression of RPE-related genes RPE65 and BEST1, and ZO-1 constituting tight junction was examined. The results are shown in FIG. 5.

RPE65:Mouse-anti RPE65, Millipore cat #MAB5428

BEST1:Live Best1::EGFP Transgenic Reporter

ZO-1:Rabbit anti-ZO-1, Invitrogen cat #61-7300

Reprogramming Media Compositions

Phase 1 Medium:

TABLE 1

| | | |
|---|---|---|
| DMEM/F12 + Glutamax ^{™} | 85% | Life Technologies |
| | | (10565-018) |
| 15% Knockout ^{™} Serum | 15% | Life Technologies |
| Replacement | | (10828-010) |
| Nonessential Amino | 1:100 | Thermo Fisher |
| Acids | | (11140-050) |
| N-2 Supplement | 1:100 | Life Technologies |
| | | (17502-048) |
| Penicillin/Streptomycin | 1:100 | Thermo Fisher |
| 100x | | (15140-122) |
| 1000X 2-Mercaptoethanol | 1:1000 | Life Technologies |
| | | (21985-023) |
| bFGF (Human | [10 ng/mL] | Wako (064-05381) |
| Recombinant) | (optional) | |

Phase 2 Medium:

TABLE 2

| | | |
|---|---|---|
| DMEM/F12 + Glutamax ™ | 85% | Life Technologies |
| | | (10565-018) |
| 15% Knockout ™ Serum | 15% | Life Technologies |
| Replacement | | (10828-010) |
| Nonessential Amino | 1:100 | Thermo Fisher |
| Acids | | (11140-050) |
| N-2 Supplement | 1:100 | Life Technologies |
| | | (17502-048) |
| Penicillin/Streptomycin | 1:100 | Thermo Fisher |
| 100x | | (15140-122) |

Phase 3 Medium:

TABLE 3

| | | |
|---|---|---|
| DMEM low glucose | 70% | Sigma (D6046) |
| F12 Ham | 30% | Sigma (N6658) |
| 100X Glutamax ™ | 1:100 | Life Technologies |
| Supplement | | (35050-061) |
| B-27 ™ Supplement | 1:50 | Life Technologies |
| | | (17502-044) |
| Penicillin/Streptomycin | 1:100 | Thermo Fisher |
| 100x | | (15140-122) |
| SB431542 | [0.5 μM] | Life Technologies |
| | | (21985-023) |
| bFGF (Human | [10 ng/mL] | Wako (064-05381) |
| Recombinant) | | |

### Example 2: Induction of RPE Cells (CRX)

Exogeneous factor with CRX and without CRX, which were introduced in the same manner as in Example 1, were compared. The number of colony formation (per 1 well) in a 6-well plate, of iRPE colonies formed on Day 9, were counted at the end of Phase1 of reprogramming gene overexpression. The results are shown in the following Table.

TABLE 4

| | Well 1 | Well 2 | Well 3 | Total | Average |
|---|---|---|---|---|---|
| With CRX | 58 | 59 | 62 | 179 | 60 |
| Without CRX | 10 | 9 | 9 | 28 | 9 |

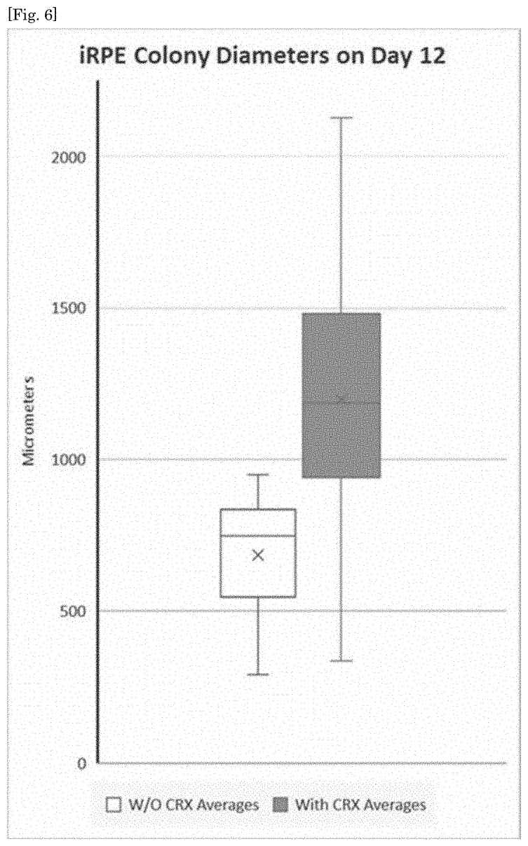

The size distribution of each colony was examined on day 12 after gene transfer. The results are shown in FIG. 6. The diameter of the colony was used as an index. Day 12 after gene transfer is in the initial stage of Phase 2.

While RPE cell was induced from the fibroblast even in the absence of CRX, the induction was more effective in the presence of CRX both in the number and size thereof. Addition of CRX is optional, but CRX induced far more colonies by number and far more proliferative colonies, measured by diameter.

### Example 3: Induction of RPE Cells (Chetomin/Nicotinamide)

The effect of treating with Chetomin and Nicotinamide in the later stage of Phase 2 was examined. In the same manner as in Example 1 except that Chetomin and Nicotinamide were added, RPE cell was induced. The results are shown in FIG. 8. Stronger dye deposition was confirmed when the both compounds were added. From these results, it was found that treatment with Chetomin and Nicotinamide at a certain stage in the process of inducing RPE cells from somatic cells was effective.

### Example 4; Transplantation of iRPE Cell of the Present Invention to Rat Eye

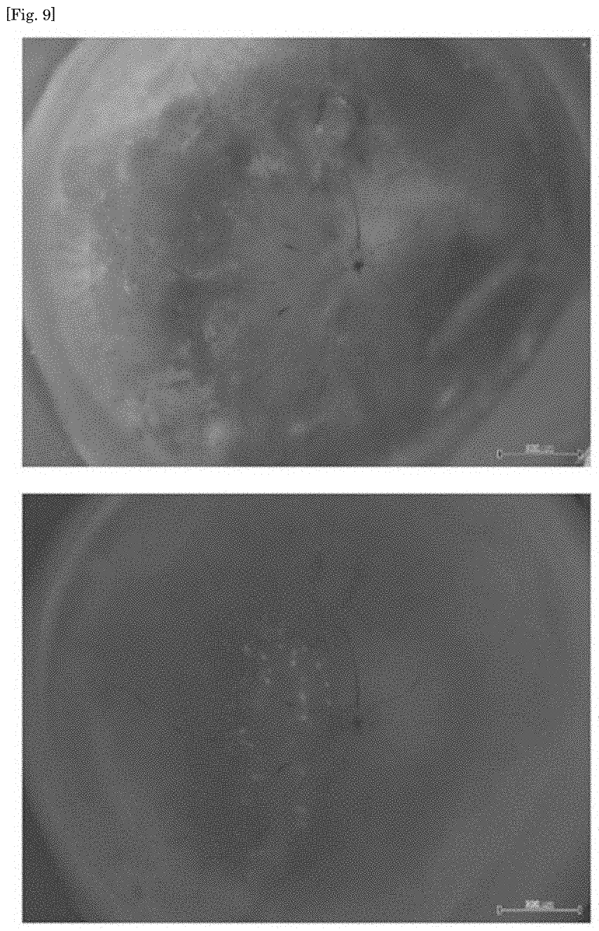

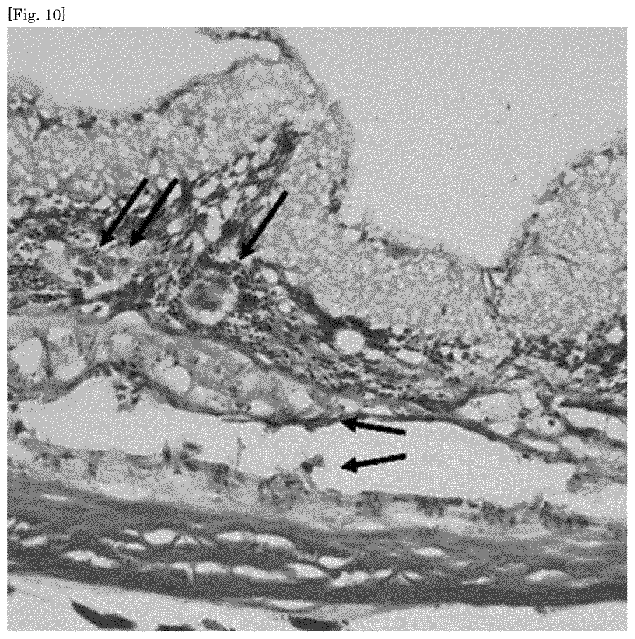

The stably-cultured human iRPE cells prepared in Example 1 were transplanted into the eye of an immunocompromised albino rat. Expression of BEST1 live reporter was examined at 4.5 months after transplantation (FIG. 9). Several large areas of the transplanted human iRPE cells expressed the BEST1::EGFP live maturation reporter gene transfer. Similarly, at 4.5 months after transplantation, a tissue section was prepared and stained with hematoxylin and eosin (FIG. 10). Several human iRPE cells have become pigmented cells that have grown, matured, and interfaced with neural retina, including integration and sheet formation in the correct RPE-layer.

BEST1: Live Best1::EGFP Transgenic Reporter

Hematoxylin and Eosin staining: Standard Practice

### INDUSTRIAL APPLICABILITY

The present invention drastically reduces the costs of patient-specific autologous RPE cells to enable a realistic cell product for regular medical and research applications. This application is based on a patent application No. 2020-033848 filed in Japan (filing date: Feb. 28, 2020), the contents of which are incorporated in full herein.

1. A method for producing a retinal pigment epithelial cell, comprising a step of introducing, as exogeneous factors,
an MITF (Microphthalmia-Associated Transcription Factor) gene or an expression product thereof,
an OTX2 (Orthodenticle homeobox 2) gene or an expression product thereof,
a LIN28 gene or an expression product thereof, and
an L-MYC gene or an expression product thereof
into a mammalian somatic cell.

2. The method according to claim 1, further comprising a step of introducing a CRX (cone-rod homeobox) gene or an expression product thereof as the exogeneous factor.

3. The method according to claim 1 or 2, comprising 3 stages of a period of overexpressing the introduced exogeneous factors in the somatic cell to yield genomic plasticity (phase 1), a period of converting the somatic cell identity to an RPE cell (phase 2), and a period of maturing the converted RPE cell (phase 3).

4. The method according to claim 3, comprising using a medium comprising bFGF and 2-ME in phase 1.

5. The method according to claim 3, comprising using a medium comprising Chetomin and Nicotinamide in a part of the period of phase 2.

6. The method according to claim 3, comprising using a medium comprising SB431542 and bFGF in phase 3.

7. The method according to claim 1, wherein the somatic cell is a human fibroblast.

8. An RPE cell produced by the method according to claim 1.

9. An RPE cell derived from a mammalian somatic cell, and comprising, as exogeneous factors,
an MITF gene or an expression product thereof,
an OTX2 gene or an expression product thereof,
a LIN28 gene or an expression product thereof, and
an L-MYC gene or an expression product thereof.

10. The cell according to claim 9, further comprising a CRX gene or an expression product thereof as the exogeneous factor.

11. The cell according to claim 9, wherein the somatic cell is a human fibroblast.

12. A reagent for directly producing a retinal pigment epithelial cell from a somatic cell, comprising
an MITF gene or an expression product thereof,
an OTX2 gene or an expression product thereof,
a LIN28 gene or an expression product thereof, and
an L-MYC gene or an expression product thereof.

13. The reagent according to claim 12, further comprising a CRX gene or an expression product thereof.

14. The reagent according to claim 12, wherein the somatic cell is a human fibroblast.
